(19) United States
(12) Patent Application Publication
(10) Pub. No.: US 2021/0275385 A1
(43) Pub. Date: Sep. 9, 2021

(54) ADJUSTABLE MASSAGE SEAT

(71) Applicant: FANG-WEI CHIOU, HSINCHU CITY (TW)

(72) Inventor: FANG-WEI CHIOU, HSINCHU CITY (TW)

(21) Appl. No.: 16/904,723

(22) Filed: Jun. 18, 2020

(30) Foreign Application Priority Data
Mar. 6, 2020 (TW) 109202512

(51) Int. Cl. A61H 7/00; A61H 1/00

(52) U.S. Cl. CPC A61H 7/002; A61H 1/005; A61H 2201/0207; A61H 2201/1692; A61H 2201/1685; A61H 2201/1695; A61H 2201/0149

(57) ABSTRACT

An adjustable massage seat includes a seat having a first connection layer laid thereon, and at least one massage knob having at least one connection member disposed thereon. The connection member can be a double-hook-sided touch fasteners to attach with the massage knob by a hook side thereof, so that the massage knob can be connected on arbitrary position of the first connection layer by the other hook side of the connection member. A vibration device or a heating device can be disposed on the massage knob to provide more massage effect. According to the present invention, the position of the massage knob disposed on the seat can be adjusted freely, to effectively relieve user's symptom upon user's requirement in massage position.

This application claims priority for Taiwan patent application no. 109202512 filed on 6 Mar. 2020, the content of which is incorporated by reference in its entirely.

### BACKGROUND OF THE INVENTION

### 1. Field of the Invention

The present invention relates to a massage application technology, and more particularly to an adjustable massage seat capable of freely changing massage position.

### 2. Description of the Related Art

As the social environment develops faster and faster, the rhythm of modern people's lives has also accelerated because the affairs to be handled have also increased relatively, and it causes that most modern people are under tense feelings. However, tense feelings also easily affect physiological conditions, such as tight muscles and backache. Therefore, most people select massage to relax tight muscles, circulate qi and blood and stretch muscle; at the same time, massage can also help people slow down the pace and soothe the tension in the mind.

However, due to the busy affairs, modern people do not have much time for massage; many manufacturers find the fact that most office workers need to sit in chairs for long-time working, and research reports indicate that there are multiple acupuncture points on the human hips, such as huan tiao acupuncture point, and these acupuncture points can help relieve soreness in waist and legs. Because of above-mentioned factors, various manufacturers have begun to develop a variety of massage devices for people who have been sitting for a long time, these massage devices include massage rollers, massage cushions and seats with massage knobs, and office workers who sit for a long time can place the massage devices on the office chairs to massage muscles while working.

But it is worth noting that all of the commercially available massage cushions or massage seats use the massage knobs fixedly and immovably disposed therein, but people's heights and body shapes are different and the locations of the acupuncture points are also different and varied from individual to individual, so many standard massage cushions or massage seats cannot meet the needs of people's requirements in massage points.

If a customized massage seat capable of adjusting a position of the massage knob freely upon different user's requirement can be developed, it is really a good news for each user.

Therefore, the present invention purposes an adjustable massage seat to solve the above-mentioned conventional problems.

### SUMMARY OF THE INVENTION

An objective of the present invention is to provide an adjustable massage seat capable of freely adjusting a position of a massage knob disposed on the seat, so as to effectively perform massage upon different user's requirement in massage position.

Another objective of the present invention is to provide an adjustable massage seat capable of vibrating or heating a user's desired massage position, to improve overall massage effect and further improve symptom relief.

In order to achieve the objectives, the present invention provides an adjustable massage seat. The adjustable massage seat includes a seat having a first connection layer laid thereon, and at least one massage knob having at least one connection member disposed thereon. The massage knob can be freely connected to the first connection layer by the connection member.

In this embodiment, the massage knob is a triangular massage knob, a rectangular massage knob, a circular massage knob, a wavy massage knob or a trapezoidal massage knob.

In this embodiment, the connection member is a double-hook-sided touch fasteners.

In this embodiment, the first connection layer is a velvet connection layer, a non-woven fabric connection layer or a linen connection layer.

In this embodiment, the seat further comprises a vibration device disposed therein.

In this embodiment, the massage knob comprises a main body and a second connection layer enclosing the main body.

In this embodiment, the main body of the massage knob includes a vibration device disposed therein.

In this embodiment, the massage knob includes a heating device disposed in the main body and connected to a plurality of heating pads, and the heating device can control the plurality of heating pads to heat. The plurality of heating pads are disposed around to the main body between the second connection layer.

In this embodiment, the main body can be an industrial foam main body.

In this embodiment, the second connection layer can be a velvet connection layer, a non-woven fabric connection layer or a linen connection layer.

### BRIEF DESCRIPTION OF THE DRAWINGS

The structure, operating principle and effects of the present invention will be described in detail by way of various embodiments which are illustrated in the accompanying drawings.

FIG. 1 is an exploded view of an adjustable massage seat of the present invention.

FIG. 2 is an exploded view of a massage knob of the present invention.

FIG. 3 is a schematic view of a usage status of an adjustable massage seat of the present invention.

FIG. 4 is a schematic view of another usage status of the adjustable massage seat of the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise” and variations such as “comprises” or “comprising”, will be acknowledged to imply the inclusion of stated elements but not the exclusion of any other elements.

The adjustable massage seat of the present invention can allow user to freely adjust the position of the massage knob disposed on the seat, to massage different position upon different user's requirement in massage position, thereby significantly improving massage effect.

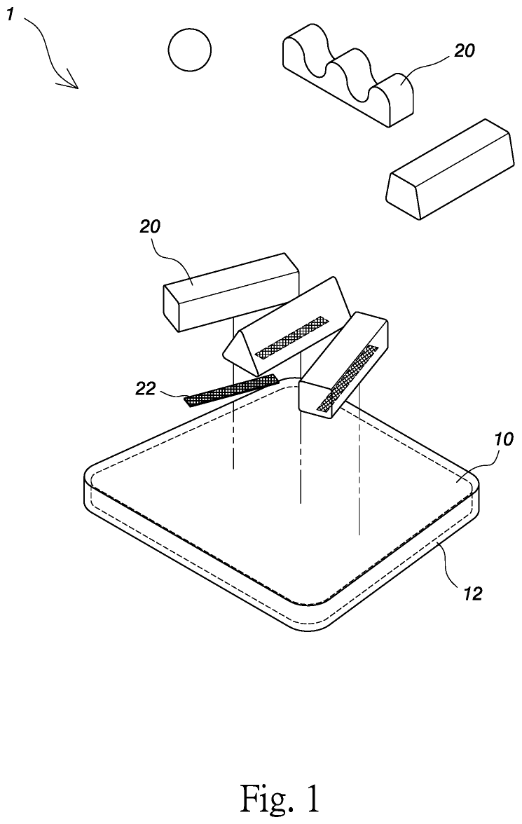

For complete understanding structural design and effect of the present invention, the embodiment of the present invention is illustrated in detail in following paragraphs. Please refer to FIG. 1. An adjustable massage seat 1 includes a seat 10 and at least one massage knob 20, the seat 10 includes a first connection layer 12 lain thereon, the first connection layer 12 can be made by the material which can be attached with a hook side of touch fasteners, for example, the first connection layer 12 can be a velvet connection layer, a non-woven fabric connection layer or a linen connection layer. In this embodiment, the number of the at least one massage knob 20 can be multiple, and the massage knobs 20 can include different shapes of massage knobs 20, such as, a triangular massage knob, a rectangular massage knob, a circular massage knob, a wavy massage knob, or a trapezoidal massage knob. Each massage knob 20 has at least one connection member 22 disposed thereon, and a user can connect the massage knob 20 in any position on the seat 10 through the connection member 22 of the massage knob 20 upon the user's requirement.

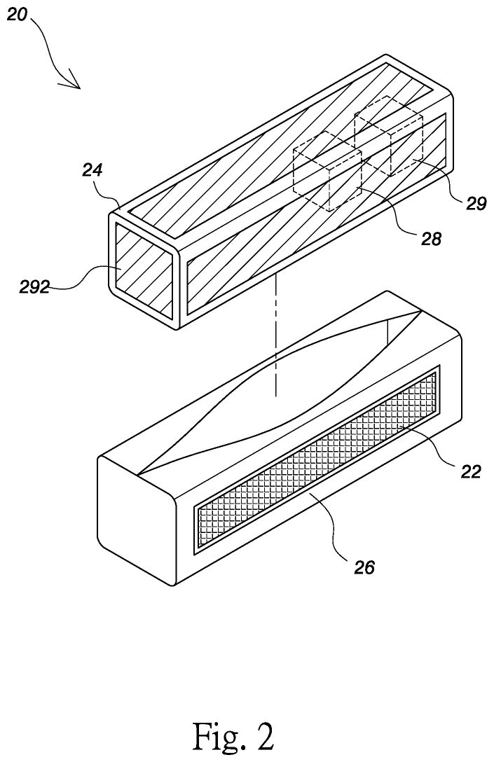

For further understanding of the technical features of the adjustable massage seat 1 of the present invention, the operation of connecting the massage knob 20 on the seat 10 through the connection member 22 and the structure of the massage knob 20 are illustrated in detail in following paragraphs with reference to FIGS. 1 and 2. The massage knob 20 includes a main body 24 and second connection layer 26; in this embodiment, the main body 24 can be an industrial foam main body. The second connection layer 26 encloses the main body 24; for example, the second connection layer 26 can be made by material which can be attached with the hook side of touch fasteners, for example, the second connection layer 26 can be a velvet connection layer, a non-woven fabric connection layer or a linen connection layer. In this embodiment, the connection member 22 can be a double-hook-sided touch fasteners, and one of the hook sides of the connection member 22 is attached with the surface of the massage knob 20, that is, attached with the second connection layer 26; the other of the hook sides of the connection member 22 is attached with the seat 10, so that the massage knob 20 can be attached on or removed from seat 10 freely.

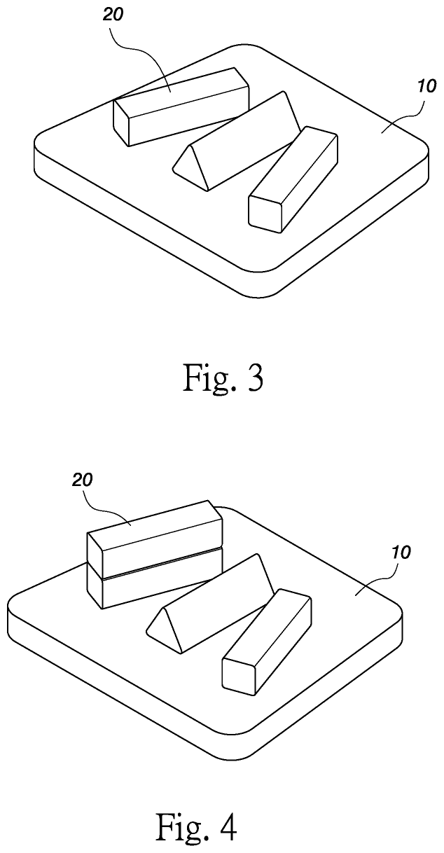

Please refer to FIG. 3. The main body 24 of the massage knob 20 can include a vibration device 28 disposed therein and configured to enable the massage knob 20 to vibrate, so as to improve massage effect. In an embodiment, the vibration device 28 can be disposed in the seat 10, as long as the adjustable massage seat 1 can generate vibration during massage operation; the vibration device 28 of the present invention is not limited to dispose in the massage knob 20. The main body 24 of the massage knob 20 can include a heating device 29 disposed therein, and the heating device 29 is electrically connected to and disposed around the outer edge of the main body 24. A plurality of heating pads 292 are disposed between the second connection layer 26, and the heating device 29 can control the plurality of heating pads 292 to heat, so as to provide heat to improve massage effect during massage operation.

According to the above-mentioned structures of the adjustable massage seat of the present invention, the usage status of the adjustable massage seat of the present invention will be described in following paragraphs with reference to FIGS. 2 and 3. When in use, the user can adjust the position of the massage knob 20 disposed on the seat 10 upon the user's body shape or desired massage position, and then turn on the vibration device 28 and the heating device 29 in the massage knob 20 after the adjustment, so as to improve the massage effect of the adjustable massage seat of the present invention. In this embodiment, the connection member 22 can be a double-hook-sided touch fasteners, the first connection layer 12 of the seat 10 and the second connection layer 26 of the massage knob 20 can be connected to the touch fasteners, so that the connection member 22 can be attached with or removed from the seat 10 or the massage knob 20 freely, and the adjustable massage seat of the present invention can have high overall variability.

Please refer to FIGS. 2 and 4. Since the second connection layer 26 on the surface of the massage knob 20 is made by material capable of being connected to the connection member 22, the massage knobs 20 can be stacked with and connected to each other to increase height of combination of the massage knobs 20, so as to strengthen intensity of massage effect.

According to the above-mentioned contents, the position of the massage knob disposed on the seat of the present invention can be adjusted freely upon the user's requirement in massage position, to effectively perform massage on massage position for different user, so the massage effect of the adjustable massage seat of the present invention can be significantly improved. Furthermore, the adjustable massage seat of the present invention can vibrate or heat the user's desired massage position, thereby improving the overall massage effect and symptom relief.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. An adjustable massage seat, comprising:
a seat comprising a first connection layer laid thereon; and
at least one massage knob comprising at least one connection member disposed thereon, wherein the at least one massage knob is freely connected to the first connection layer by the at least one connection member.

2. The adjustable massage seat according to claim 1, wherein the at least one massage knob is a triangular massage knob, a rectangular massage knob, a circular massage knob, a wavy massage knob or a trapezoidal massage knob.

3. The adjustable massage seat according to claim 1, wherein the connection member is a double-hook-sided touch fasteners.

4. The adjustable massage seat according to claim 1, wherein the first connection layer is a velvet connection layer, a non-woven fabric connection layer, or a linen connection layer.

5. The adjustable massage seat according to claim 1, wherein the seat further comprises a vibration device disposed therein.

6. The adjustable massage seat according to claim 1, wherein the at least one massage knob comprises a main body and a second connection layer enclosing the main body.

7. The adjustable massage seat according to claim 6, wherein the main body of the at least one massage knob comprises a vibration device disposed therein.

8. The adjustable massage seat according to claim 6, wherein the at least one massage knob comprises a heating device disposed in the main body thereof, and the heating device is connected to a plurality of heating pads and configured to control the plurality of heating pads to heat, and the plurality of heating pads are disposed around the main body and between the second connection layer.

9. The adjustable massage seat according to claim 6, wherein the main body is an industrial foam main body.

10. The adjustable massage seat according to claim 6, wherein the second connection layer is a velvet connection layer, a non-woven fabric connection layer or a linen connection layer.
